(19) United States
(12) Patent Application Publication
(10) Pub. No.: US 2011/0047651 A1
(43) Pub. Date: Feb. 24, 2011

(54) TRANSFORMATION AND ENGINEERED TRAIT MODIFICATION IN MISCANTHUS SPECIES

(75) Inventors: Dean Engler, Moraga, CA (US); Jianxin Chen, Mountain View, CA (US)

Correspondence Address: Mendel Biotechnology, Inc., 3935 Point Eden Way, Hayward, CA, 94545, US

(73) Assignee: Mendel Biotechnology, Inc., Hayward, CA (US)

(21) Appl. No.: 12/988,789

(22) PCT Filed: Apr. 22, 2009
(86) PCT No.: PCT/US09/41424

§ 371 Date: Oct. 20, 2010

Related U.S. Application Data
(60) Provisional application No. 61/047,391, filed on Apr. 23, 2008.

(51) Int. Cl. A01H 1/06; A01H 5/00; A01H 5/10; C12N 5/10

(52) U.S. Cl. 800/294; 800/298; 800/300; 435/419

(57) ABSTRACT

Methods and compositions for the efficient transformation of Miscanthus are provided. The method involves infection with Agrobacterium, particularly those comprising a binary vector. In this manner, any gene or nucleotide sequence of interest can be introduced into the Miscanthus plant. The transformed gene or nucleotide sequence of interest will be flanked by at least one T-DNA border and present in the transformed Miscanthus in low copy number. Transformed Miscanthus, cells, tissues, plants, and seed are also provided.

### FIELD

Methods and compositions for the transformation of Miscanthus, particularly methods for transformation utilizing Agrobacterium are described.

### BACKGROUND

Miscanthus is a monocot C4 grass genus of the Saccharum complex comprising approximately fourteen species. Miscanthus is from the family Poaceae, tribe andropogoneae, subtribe saccharinae. Miscanthus has a basic chromosome number of 19, with diploid and tetraploid species common Common species are sinensis, sacchariflorus, floridulus, transmorrisonensis, condensatus, and include the hybrid form Miscanthus×giganteus, a triploid resulting from a cross between the diploid sinensis and the tetraploid sacchariflorus.

Recently, Miscanthus has gained attention as a potential biofuel crop because of its ability to yield high amounts of high quality lignocellulosic material. However, nearly all advanced research and development of Miscanthus as a biofuel feedstock has focused on only one genotype of M.×giganteus. M.×giganteus is characterized by relatively high yields and low moisture content at harvest. M.×giganteus has also demonstrated high water and nitrogen use efficiencies as well as low susceptibility to pests and diseases. These traits make Miscanthus especially promising as a sustainable biofuel feedstock crop.

Almost all previous breeding work on M. sinensis has been for the development of ornamental varieties for gardens. Other species of Miscanthus have received little or no attention from scientists and horticulturalists. Thus, any work that results in genetic improvement of Miscanthus for use as a biofuel would likely be a novel contribution. Until recently, genetic improvement of Miscanthus has been carried out by traditional plant breeding methods. Advances in tissue culture and transformation technologies have resulted in the production of transgenic Miscanthus sacchariflorus utilizing microprojectile bombardment as the transformation system (Yi et al. (2004) High Technol. Lett.). However, the use of particle bombardment as a transformation vehicle has its disadvantages. For example, using bombardment transformation, many copies of the transferred sequence are routinely integrated into the targeted genome. These integrated copies are often rearranged and mutated. Further, the integrated sequences are often unstable due to the insertion point (Casa et al (1993) Proc. Natl. Acad. Sci. USA 90:11212-11216).

In contrast to particle bombardment transformation, it has been demonstrated that Agrobacterium-mediated transformation results in a greater proportion of stable, low-copy (i.e., one or two) number transgenic events than does bombardment transformation (Ishida et al. (1996) Nature Biotechnol. 14:745-750; Zhao et al. (1998) Maize Genet. Coop Newslett. 72:34-37), offers the possibility of transferring larger DNA segments into recipient cells (Hamilton et al. (1996) Proc. Natl. Acad. Sci. USA 93:9975-9979), and is highly efficient (Ishida et al. (1996) Nature Biotechnol. 14:745-750; Zhao et al. (1998) Maize Genet. Coop. Newslett. 72:34-37). Therefore, it is advantageous to develop a transgenic plant using Agrobacterium-mediated transformation. Gene transfer by means of engineered Agrobacterium strains has become routine for most dicotyledonous plants.

However, gene transfer by means of Agrobacterium strains for monocotyledonous plants such as Miscanthus (see, for example, Cheng et al. (2004) Plant 40(1):3145, and the references cited therein; also see Shrawat et al (2006) Plant Biotechnol. J. 4:575-603, and the references cited therein) is limited due to the recalcitrance of monocotyledonous plants with respect to interaction with Agrobacterium species.

To date, scientists have advocated that Agrobacterium-mediated transformation be applied to the production of transgenic Miscanthus species (Juvik et al. (2007) “Miscanthus Breeding and Improvement”, at “4^{th} Annual Open Symposium on Biomass Feedstocks for Energy Production in Illinois”, University of Illinois at Urbana-Champaign), and have made attempts to produce Miscanthus plants using such a method (miscanthus.uiuc.edu/index.php/researchers/dr-jack-juvik/sma). Despite years of attempts, however, the production of a transgenic Miscanthus plant utilizing Agrobacterium has not yet been achieved.

Accordingly, there is needed an efficient method for the transformation of Miscanthus wherein stable transformation of desired sequences can be obtained, particularly using an Agrobacterium-mediated transformation method.

### SUMMARY

Methods and compositions for the efficient transformation of Miscanthus are described. The method involves the use of bacteria belonging to the genus Agrobacterium, particularly those comprising a binary vector. In this manner, any gene of interest, in fact any sequence, can be introduced into the Miscanthus plant. The transferred gene will be flanked by at least one T-DNA border and present in the transformed Miscanthus in low copy number.

Transformed Miscanthus cells, tissues, plants, and seed are also provided. Such transformed compositions are characterized by the presence of one or more T-DNA borders and a low copy number of the transferred gene. Transformed compositions also encompass sterile Miscanthus plants as well as regenerated, fertile transgenic Miscanthus plants, transgenic seeds produced therefrom, in T1 and subsequent generations.

The invention also pertains to transgenic Miscanthus plants and methods for their preparation, where an embryogenic callus is first selected for its ability to be grown into a mature Miscanthus plant. This “ecallus” is contacted with agrobacteria comprising a plasmid of interest, the bacteria and the ecallus are co-cultivated to produce transformed ecallus, and the latter is then grown into a transgenic Miscanthus plant. The selection of ecallus for its regenerative ability may be accomplished using specific morphological characteristics or by an analysis of chlorophyll synthesis after exposure of the ecallus to light.

### DETAILED DESCRIPTION

I. Definitions

Various terms are used throughout the specification and statements. Unless otherwise specified, these terms are defined as set forth below.

“Sustainably regenerable callus” as used herein means a callus that is sufficiently regenerable following induction that, once transformed, it can be regenerated into whole plants.

“Transformation” as used herein is the genetic alteration of a cell resulting from the uptake, stable integration in the cell's genome, and expression of foreign genetic material (DNA).

“T-DNA” as used herein is any sequence that can be utilized by Agrobacterium as border sequences for initiation and/or termination of DNA transfer to plants, and all sequences between such border sequences, such as sequences from Agrobacterium, or related sequences from plants, defined by Romens as P-DNA (see Romens et al. (2005) Plant Physiol. 139:1338-1349).

“Regenerate” refers to the creation of mature plants from plant tissue, such as embryogenic callus, or possibly from early stage embryos, and “regenerative capability” refers to the ability to give rise to whole plants.

“Plant”, “transformed plant”, and “transgenic plant”, may each refer to parts, tissues, or individual cells of a plant. These terms also include plant material that can be regenerated into a mature plant, including but not limited to protoplasts or callus tissue.

A “mature plant” is a plant in which normal development of all vegetative and reproductive organs that is generally associated with the species of that plant has taken place.

II. Description

Compositions and methods for the efficient transformation of Miscanthus are provided. The transformed Miscanthus plants are characterized by containing transferred nucleic acid such as a transferred gene or genes of interest flanked by at least one T-DNA border inserted within the genome of the Miscanthus plants. The plants are normal in morphology and may be fertile, depending on the species of Miscanthus. Generally, the transformed plants contain a single copy of the transferred nucleic acid with no notable rearrangements. Alternatively, the transferred nucleic acid of interest is present in the transformed Miscanthus in low copy numbers. By low copy number is intended that transformants include no more than five (5) copies of the transferred nucleic acid, preferably, no more than three (3) copies of the transferred nucleic acid, more preferably, fewer than three (3) copies of the transferred nucleic acid, even more preferably, (1) copy of the transferred nucleic acid. The transferred nucleic acid will include at least one T-DNA border sequence.

The methods described herein rely upon the use of Agrobacterium-mediated gene transfer. Agrobacterium-mediated gene transfer exploits the natural ability of Agrobacterium tumefaciens or Agrobacterium rhizogenes to transfer DNA into plant chromosomes. Agrobacterium is a plant pathogen that transfers a set of genes encoded in a region called T-DNA of the Ti plasmid (Agrobacterium tumefaciens) or Ri plasmid (Agrobacterium rhizogenes) into plant cells at wound sites. The typical result of gene transfer is a tumorous growth called a crown gall in which the T-DNA is stably integrated into a host chromosome. The ability to cause crown gall disease can be removed by deletion of the genes in the T-DNA (e.g., disarmed T-DNA) without loss of DNA transfer and integration. The DNA to be transferred is attached to border sequences that define the end points of an integrated T-DNA.

Gene transfer by means of engineered Agrobacterium strains has become routine for most dicotyledonous plants and for some monocotyledonous plants (see, for example, Cheng, et al. (2004) Plant:40(1):3145, and the references cited therein; also see Shrawat, et al (2006) Plant Biotechnol. J. 4, pp. 575-603, and the references cited therein). However, there are no reports to date of producing transformed Miscanthus by means of Agrobacterium-mediated transformation.

The Agrobacterium strain utilized in the methods described herein is modified to contain a gene or genes of interest, or a nucleic acid to be expressed in the transformed cells. The nucleic acid to be transferred is incorporated into the T-region and is flanked by at least one T-DNA border sequence. A variety of Agrobacterium species are known in the art particularly for dicotyledon transformation. Such agrobacteria can be used in the methods described herein. See, for example, Hooykaas (1989) Plant Mol. Biol. 13:327-336; Smith et al. (1995) Crop Sci. 35:301-309; Chilton (1993) Proc. Natl. Acad. Sci. USA 90:3119-3120; Mollony et al. (1993) Monograph Theor. Appl. Genet., NY 19:148; Ishida et al. (1996) Nature Biotechnol. 14:745-750; and Komari et al. (1996) Plant J. 10:165-174; herein incorporated by reference.

In the Ti/Ri plasmid, the T-region is distinct from the vir region whose functions are responsible for transfer and integration. Binary vector systems have been developed where the manipulated disarmed T-DNA carrying foreign DNA and the vir functions are present on separate plasmids. In this manner, a modified T-DNA region comprising foreign DNA (the nucleic acid to be transferred) is constructed in a small plasmid which replicates in E. coli. This plasmid may be transferred conjugatively in a tri-parental mating or may be transferred by alternative means such as by electroporation into A. tumefaciens or rhizogenes which contains a compatible plasmid-carrying virulence gene. The vir functions are supplied in trans to transfer the T-DNA into the plant genome. Such binary vectors are useful in the practice of the present methods and in the production of compositions described herein.

Super-binary vectors can also be used in the present methods and in the production of compositions described herein. See, for example, U.S. Pat. No. 5,591,616 and EPA 0604662A1, herein incorporated by reference. Such a super-binary vector has been constructed containing a DNA region originating from the virulence region of Ti plasmid pTiBo542 (Jin et al. (1987) J. Bacteriol. 169:4417-4425) contained in a super-virulent Agrobacterium tumefaciens A281 exhibiting extremely high transformation efficiency (Hood et al. (1984) Biotechnol. 2:702-709; Hood et al. (1986) J. Bacteriol. 168:1283-1290; Komari et al. (1986) J. Bacteriol. 166:88-94; Jin et al. (1987) J. Bacteriol. 169:4417-4425; Komari T. (1989) Plant Sci. 60:223-229; ATCC Accession No. 37394).

As will be evident to one of skill in the art, now that a method has been provided for stable transformation of Miscanthus, any nucleic acid of interest can be used in the methods described herein. For example, a Miscanthus plant can be engineered to express disease and insect resistance genes, genes to increase yield or biomass, genes to improve tolerance to a range of abiotic stresses (including, but not limited to, drought, heat, cold and freezing), genes to modulate lignin content, genes to confer male and/or female sterility, antifungal, antibacterial or antiviral genes, and the like. Likewise, the method can be used to transfer any nucleic acid to control gene expression. For example, the nucleic acid to be transferred could encode an antisense oligonucleotide.

General categories of genes of interest include, for example, those genes involved in regulation of gene expression, such as members of the zinc finger family, the AP2 family, the MADS family and including any of the other families listed below, those involved in signaling, such as kinases and phosphatases, and those involved in housekeeping, such as enzymes of anabolic and catabolic pathways, and heat shock proteins. More specific categories of transgenes, for example, include genes encoding important agronomic traits, such as insect resistance, disease resistance, nematode resistance, herbicide resistance, sterility, grain characteristics, flowering time, inherent yield, photosynthetic capacity, drought tolerance, water use efficiency, nutrient use efficiency (e.g., nitrogen, phosphorous), and genes encoding morphological properties, such as root growth and branching, leaf extension, trichome growth and development, stomatal specification, flower fate, and meristem fate.

Other categories of genes of interest may further include members of the following families: the MYB transcription factor family; the WRKY protein family; the ankyrin-repeat protein family; the homeobox (HB) protein family; the CAAT-element binding proteins; the squamosa promoter binding proteins (SPB); the NAM protein family; the HLH/MYC protein family; the DNA-binding protein (DBP) family; the bZIP family of transcription factors; the Box P-binding protein (the BPF-1) family; the high mobility group (HMG) family; the scarecrow (SCR) family; the GF14 family; the polycomb (PCOMB) family; the teosinte branched (TEO) family; the ABI3 family; the EIL family; the AT-HOOK family; the S1FA family; the bZIPT2 family; the YABBY family; the PAZ family; a family of miscellaneous (MISC) transcription factors including the DPBF family and the SPF1 family; the GARP family, the TUBBY family, the heat shock family, the ENBP family; the RING-zinc family, the PDBP family, the PCF family; the SRS (SHI-related) family; the CPP (cysteine-rich polycomb-like) family; the ARF (auxin response factor) family; the SWI/SNF family; the ACBF family; the PCGL (CG-1 like) family; the ARID family; the Jumonji family; the bZIP-NIN family; the E2F family; and, the GRF-like family.

Commercially important “output” traits such as oil, starch and protein content or composition can be genetically altered using the transformation methods described herein. Modifications include reduced or increased cellulose content, reduced or increased hemicellulose content, reduced or increased lignin content. The specific composition of hemicellulose can also be modified, for example by changing the relative content of C5 sugars such as xylose or arabinose, and by modifying linkages of hemicellulose to organic acids such as ferulic acid. Modifications also include the relative amounts of syringyl, guaicyl, and other forms of lignin based on incorporation of lignin precursor substrates. Additional modifications include increasing content of oleic acid, saturated and unsaturated oils, increasing levels of lysine and sulfur and providing essential amino acids, and also modification of starch.

Insect resistance genes may encode resistance to pests that cause significant yield reductions. For example, genes from the microorganism Bacillus thuringiensis encode toxic proteins that have been isolated, characterized and successfully used to lessen ECB infestation (U.S. Pat. No. 5,366,892, Foncerrada et al. Gene Encoding a Coleopteran-active Toxin). Other examples of genes useful in insect resistance include those encoding secondary metabolites and plant toxins.

Herbicide resistance traits may include genes coding for resistance to herbicides which act to inhibit the action of acetolactate synthase (ALS), in particular, the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and Geneticin®, and the ALS gene encodes resistance to the herbicide chlorsulfuron.

Herbicide resistance traits may also include genes that confer resistance to herbicides such as glyphosate or sulfonamide. Resistance to glyphosate herbicides can be obtained by using genes coding for the mutant target enzymes, 5-enolpyruvylshikimate-3-phosphate synthase (EPSPS) (see, for example, WO 01/66704). Resistance to sulfonamide can be obtained by using bacterial genes that encode a protein having sulfonamide-insensitive dihydropteroate synthase (DHPS) activity (sul proteins), and expressing the protein in plant mitochondria (see, for example, U.S. Pat. No. 6,121,513).

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development. In addition, genes encoding factors that modify flowering time, or which repress conversion of a plant meristem from vegetative to flowering identity, are also usefully expressed.

Commercial traits can also be encoded on a gene or genes which could modify for example, cell wall composition or linkage between lignin and other cell wall components, for increased ethanol (or other biofuel) production, or provide expression of proteins. Another important commercial use of transformed plants is the production of polymers and bioplastics, such as described in U.S. Pat. No. 5,602,321, issued Feb. 11, 1997. Genes such as, B-ketothiolase, PHBase (polyhydroxyburyrate synthase) and acetoacetyl-CoA reductase (see Schubert et al. (1988) J. Bacteriol. 170) facilitate expression of polyhyroxyalkanoates (PHAs).

The compositions and methods described herein are particularly useful for the production of transgenic Miscanthus plants that are modified to exhibit traits that would be advantageous in the production of biofuel. For example, genes that can modulate biochemical pathways (e.g. for improved nutrient use efficiency, improved water use efficiency and improved photosynthetic efficiency), plant architecture (e.g. shoot number, stalk size, and height), resistance to pests and diseases, tolerance to abiotic stresses (e.g. drought tolerance, salt tolerance, and ozone tolerance), and resistance to herbicides would be useful for increasing biomass yields of Miscanthus species. Marker gene expression (e.g., herbicide or antibiotic resistance and reporter genes) would increase the efficiency of producing improved varieties of Miscanthus. In addition, genes that can modify the quality of the biomass produced by Miscanthus would be useful for improving the efficiency of conversion to fuels such as ethanol. An enormous amount of research is currently underway to advance the use of biomass for biofuel (see, for example, the website of the National Renewable Energy Laboratory on the World Wide Web: “nrel.gov/biomass”). The methods and compositions described herein can be used to advance such efforts.

For convenience, the nucleic acid to be transferred can be contained within expression cassettes. The expression cassette will generally include a transcriptional initiation region linked to the nucleic acid or gene of interest. Such an expression cassette is provided with a plurality of restriction sites for insertion of the gene or genes of interest to be under the transcriptional regulation of the regulatory regions.

The transcriptional initiation region, the promoter, may be native or homologous or foreign or heterologous to the host, or could be the natural sequence or a synthetic sequence. By foreign is intended that the transcriptional initiation region is not found in the wild-type host into which the transcriptional initiation region is introduced. As used herein a chimeric gene includes a coding sequence operably linked to transcription initiation region which is heterologous to the coding sequence.

The transcriptional cassette will include the in 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the Ti-plasmid of A. tumefaciens, such as the octopine synthase and nopaline synthase termination regions. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; Joshi et al. (1987) Nucleic Acid Res. 15:9627-9639.

Alternatively, the gene(s) of interest can be provided on another expression cassette. Where appropriate, the gene(s) may be optimized for increased expression in the transformed plant. Where mammalian, yeast, or bacterial or dicot genes are used in the invention, they can be synthesized using monocot or Miscanthus preferred codons for improved expression. Methods are available in the art for synthesizing plant preferred genes. See, for example, U.S. Pat. Nos. 5,380,831, 5,436,391, and Murray et al. (1989) Nucleic Acids Res. 17:477-498, herein incorporated by reference.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Allison et al. (1986) Virol. 154:9-20), and human immunoglobulin heavy-chain binding protein (BiP) (Macejak and Sarnow (1991) Nature 353:90-94; untranslated leader from the coat protein mRNA of alfalfa mosaic virus (AMV RNA 4) (Jobling and Gehrke (1987) Nature 325:622-625; tobacco mosaic virus leader (TMV) (Gallie et al. (1989) Molecular Biology of RNA, pages 237-256; and maize chlorotic mottle virus leader (MCMV) (Lommel et al. (1991) Virol. 81:382-385). See also, Della-Cioppa et al. (1987) Plant Physiol. 84:965-968. Other methods known to enhance translation can also be utilized, for example, introns, and the like.

The expression cassettes may contain one or more than one gene or nucleic acid sequence to be transferred and expressed in the transformed plant. Thus, each nucleic acid sequence will be operably linked to 5′ and 3′ regulatory sequences. Alternatively, multiple expression cassettes may be provided.

Generally, an expression cassette will be included which contains a selectable marker gene for the selection of transformed cells. Selectable marker genes are utilized for the selection of transformed cells or tissues. Selectable marker genes include genes encoding antibiotic resistance, such as those encoding neomycin phosphotransferase II (NPT) and hygromycin phosphotransferase (HPT) as well as genes conferring resistance to an herbicide or for an enzyme that degrades or detoxifies the herbicide in the plant before it can act. (See DeBlock et al. (1987) EMBO J. 6:2513-2518; DeBlock et al. (1989) Plant Physiol., 91:691-704; Fromm et al. (1990) Bio/Technology 8:833-839. For example, resistance to glyphosate or sulfonylurea herbicides has been obtained by using genes coding for the mutant target enzymes EPSPS and ALS. Resistance to glufosinate ammonium, bromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

Selectable marker genes which find use in the methods described herein include, but are not limited to genes encoding: neomycin phosphotransferase II (Fraley et a. (1986) CRC Critical Reviews in Plant Science 4:1-25); cyanamide hydratase (Maier-Greiner et al. (1991) Proc. Natl. Acad. Sci. USA 88:4250-4264); aspartate kinase; dihydrodipicolinate synthase (Perl et al. (1993) Bio/Technology 11:715-718); tryptophan decarboxylase (Goddijn et al. (1993) Plant Mal. Biol. 22:907-912); dihydrodipicolinate synthase and desensitized aspartade kinase (Perl et al. (1993) Bio/Technology 11:715-718); bar gene (Toki et al. (1992) Plant Physiol. 100:1503-1507 and Meagher et al. (1996) Crop Sci, 36:1367-1374); tryptophane decarboxylase (Goddijn et al. (1993) Plant Mol. Biol., 22:907-912); neomycin phosphotransferase (NPT) (Southern et al. (1982) J. Mol. Appl. Gen., 1:327-331; hygromycin phosphotransferase (HPT or HYG) (Shimizu et al. (1986) Mol. Cell Biol., 6:1074-1087); dihydrofolate reductase (DHFR) (Kwok et al. (1986) Proc. Natl. Acad. Sci. USA:83:4552-4555); phosphinothricin acetyltransferase (DeBlock et al. (1987) EMBO J. 6:2513-2518); 2,2-dichloropropionic acid dehalogenase (Buchanan-Wollatron et al. (1989) J. Cell. Biochem. 13D:330); acetohydroxyacid synthase (Anderson et al U.S. Pat. No. 4,761,373; Haughn et al. (1988) Mol. Gen. Genet. 221:266); 5-enolpyruvyl-shikimate-phosphate synthase (aroA) (Comai et al. (1985) Nature 317:741-744); haloarylnitrilase (Stalker et al. PCT appl. WO87/04181); acetyl-coenzyme A carboxylase (Parker et al. (1990) Plant Physiol. 92:1220-1225); dihydropteroate synthase (sul I) (Guerineau et al. (1990) Plant Mol. Biol. 15:127-136); 32 kD photosystem II polypeptide (psbA) (Hirschberg et al. (1983) Science 222:1346-1349); etc.

Also included are genes encoding resistance to: chloramphenicol (Herrera-Estrella et al. (1983) EMBO J. 2:987-992); methotrexate (Herrera-Estrella et al. (1983) Nature 303:209-213; Meijer et al. (1991) Plant Mol Biol. 16:807-820 (1991); hygromycin (Waldron et al. (1985) Plant Mol. Biol., 5:103-108; Zhijian et al. (1995) Plant Science 108:219-227 and Meijer et al. (1991) Plant Mol. Biol. 16:807-820); streptomycin (Jones et al. (1987) Mol. Gen. Genet. 210:86-91); spectinomycin (Bretagne-Sagnard et al. (1996) Transgenic Res., 5:131-137); bleomycin (Hille et al. (1986) Plant Mal. Biol. 7:171-176); sulfonamide (Guerineau et al. (1990) Plant Mal. Biol. 15:127-136); bromoxynil (Stalker et al. (1988) Science 242:419-423); 2,4-D (Streber et al. (1989) Bio/Technology 7:811-816); glyphosate (Shaw et al. (1986) Science 233:478-481); phosphinothricin (DeBlock et al. (1987) EMBO J. 6:2513-2518); spectinomycin (Bretagne-Sagnard and Chupeau (1996) Transgenic Res. 5:131-137).

The bar gene confers herbicide resistance to glufosinate-type herbicides, such as phosphinothricin (PPT) or bialaphos, and the like. As noted above, other selectable markers that could be used in the vector constructs include, but are not limited to, the pat gene, also for bialaphos and phosphinothricin resistance, the ALS gene for imidazolinone resistance, the HPH or HYG gene for hygromycin resistance, the EPSP synthase gene for glyphosate resistance, the Hml gene for resistance to the Hc-toxin, and other selective agents used routinely and known to one of ordinary skill in the art.

See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol., 6:2419-2422; Barkley et al. (1980) The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) PhD Thesis, University of Heidelberg; Reines et al. (1993) Proc. Natl. Acad. Sci. USA 90:1917-1921; Labow et al. (1990) Mol. Cell Biol. 10:3343-3356; Zambretti et al. (1992) Proc. Natl. Acad. Sci. USA 89:3952-3956; Bairn et al. (1991) Proc. Natl. Acad. Sci. USA 88:5072-5076; Wyborski et al. (1991) Nuc. Acids Res. 19:4647-4653; Hillenand-Wissman (1989) Topics Mol. Struc. German (Germany) Biol., 10:143-162; Degenkolb et al. (1991) Antimicrob. Agents Chemother. 35:1591-1595; Kleinschnidt et al. (1988) Biochemistry 27:1094-1104; Gatz et al. (1992) Plant J. 2:397-404; Bonin (1993) PhD Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Exp. Pharmacology, 78; Gill et al. (1988) Nature 334:721-724. Such disclosures are herein incorporated by reference.

The above list of selectable marker genes is illustrative only, and not meant to be limiting. Any selectable marker gene can be used to practice the disclosed methods and compositions.

Where appropriate, the selectable marker genes and other gene(s) and nucleic acid of interest to be transferred can be synthesized for optimal expression in Miscanthus. That is, the coding sequence of the genes can be modified to enhance expression in Miscanthus. The synthetic nucleic acid is designed to be expressed in the transformed tissues and plants at a higher level. The use of optimized selectable marker genes may result in higher transformation efficiency.

Methods for synthetic optimization of genes are available in the art. The nucleotide sequence can be optimized for expression in Miscanthus or alternatively can be modified for optimal expression in monocots. The plant preferred codons may be determined from the codons of highest frequency in the proteins expressed in Miscanthus, particularly those proteins expressed at high levels in one or more tissues of the plant. It is recognized that genes which have been optimized for expression in maize and other monocots can be used in the methods described herein. See, for example, EPA 0359472; EPA 0385962; WO 91/16432; Perlak et al. (1991) Proc. Natl. Acad. Sci. USA 88:3324-3328; and Murray et al. (1989) Nucleic Acids Res. 17:477-498. U.S. Pat. No. 5,380,831; U.S. Pat. No. 5,436,391; and the like, herein incorporated by reference. It is further recognized that all or any part of the gene sequence may be optimized or synthetic. That is, fully optimized or partially optimized sequences may also be used.

Additional sequence modifications are known to enhance gene expression in a cellular host. These include elimination of sequences encoding spurious polyadenylation signals, exon-intron splice site signals, transposon-like repeats, and other such well-characterized sequences which may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The methods described herein are useful for producing transgenic Miscanthus plants. The Miscanthus specie used in the Examples described herein is M. sinesis. However, any Miscanthus specie can be used to produce transgenic Miscanthus plants using the methods described herein. Other Miscanthus species include, for example, sacchariflorus, floridulus, transmorrisonensis, condensatus, and the hybrid form Miscanthus×giganteus, a triploid resulting from a cross between the diploid sinensis and the tetraploid sacchariflorus.

Miscanthus plants and seed are generally available at ornamental nurseries. Nurseries typically provide plants or rhizomes to ensure 100% survival after planting. Miscanthus sinensis seed used in the Examples were obtained from Jelitto Staudensamen GmbH, Am Toggraben 3, 29690, Schwarmstedt, Germany.

Miscanthus seed from fertile genotypes and/or species not available from nurseries may be obtained by collecting seed from plants in their natural habitats (e.g., Asia), or from botanical gardens or germplasm collection centers (e.g., Kew Gardens (UK)). Sterile species, rhizomes, plants or tissue culture plants can generally be obtained from nurseries or biotechnology companies (e.g., Tinplant, Germany).

To obtain seed from a fertile Miscanthus plant, rhizome or tissue culture, a second plant is generally needed for a cross and successful seed production as Miscanthus is self incompatible (apart from a report of apomixis in M. floridulus and a report of self compatibility in M. condensatus). A few seed can often be obtained from self pollination of a single plant due to incomplete incompatibility.

The methods described herein are useful for producing transgenic Miscanthus plant cells. Such cells include embryogenic callus (ecallus) which can be originated from any tissues of Miscanthus plants. Preferably, the tissue utilized in initiating ecallus is immature tissue such as immature embryos, immature inflorescences (spikelet tissue), and the basal portion of young leaves. Alternatively, the ecallus can be originated from seeds or germinated seedling tissues, anthers or other anther tissues such as filaments, microspores, mature embryos, and in principal from any other tissue of Miscanthus capable of forming ecallus.

Miscanthus ecallus can be produced from seeds, from immature portions of the inflorescences, preferably immature spikelets or from cultured immature spikelets. Initiation of embryogenic callus from seeds is done as follows:

The Miscanthus seeds are sterilized by any method know in the art such as by immersion of the seeds in 20% bleach with 0.1% triton X-100 for 20 minutes followed by rinsing with sterilized water. The sterilized seeds are then plated onto a solid medium such as “Embryogenic callus induction and growth medium” (MECG) which contains all the elements necessary for induction and growth of Miscanthus embryogenic callus. Petri dishes are convenient for this process, but any vessel can be used. It is necessary to protect the media with the seeds from desiccation. A convenient method is to seal the vessel with Saran Wrap™ or Parafilm®. The plates are incubated preferably in continuous darkness at between 22° C. and 32° C., preferably about 29° C. In these conditions, the seeds will germinate, and ecallus can be expected to be present on the germinated seedlings within 2 to 4 weeks. Subculture of the ecallus is generally done about 4 to 6 weeks after plating the seeds.

Initiation of ecallus from immature spikelets is done as follows:

Miscanthus plants are grown in pots, preferably 2 gallon pots until they produce flowering structures. When the tillers are at the stage when 5 to 6 fully open leaves are present, the top node is collected and sterilized in a manner similar to the way seeds are sterilized. Sterile spikelets are then isolated from the top node, and placed onto MECG media (see examples for media composition) as is done for seeds. Embryogenic callus can be expected to be present on the spikelets within 2 to 4 weeks. Subculture of the ecallus is done about 4 to 6 weeks after plating the immature spikelets.

Initiation of ecallus from cultured immature spikelets is done similarly to the initiation of ecallus from immature spikelets, except that prior to plating the spikelets onto MECG media, they are first plated onto “Basal Medium” (MSMO; see examples for media composition) for 2 to 4 weeks and then the cultured immature spikelets are treated the same as immature spikelets. This procedure has the advantage that more tissue can be produced prior to plating on MECG media, and thus more ecallus can be produced from a given number of immature spikelets.

The method described herein can also be used to transform cell suspensions. Such cell suspensions can be formed from any Miscanthus tissue. Preferably the tissue utilized to initiate cell suspensions is ecallus formed as described above.

After ecallus is obtained, optional culture steps may be used to increase the quantity or quality (such as the regenerability) of ecallus and select for regenerable ecallus prior to transformation. The first culture step involves culturing the ecallus or target tissue prior to the infection step on a suitable medium such as (MECG) (See Example 1). The culture period may last as long as is necessary to produce enough ecallus for transformation, yet not so long that undesirable somaclonal variation or loss of regenerability has occurred. Typically this culture period lasts from 4 months to about 1 year. In culturing ecallus, the ecallus can be routinely subcultured about every three to four weeks and preferentially selected ecallus can be transferred to fresh media for further culture steps. The ecallus is typically cultured in darkness at a temperature of 22 to 32 degrees C., preferably about 29 degrees C.

The loss of regenerability in cultured ecallus is a potential problem that must be avoided in order to be able to regenerate whole plants following a transformation protocol. This is accomplished by visually selecting and transferring only the regenerable type of ecallus while discarding the non-regenerable ecallus at each sub-culture step. One means for visual identification of regenerable ecallus is by use of callus morphology. The types of monocot ecallus morphology which have retained the capacity for regeneration are well known in the art, however use of these methods of visual selection may be inadequate to maintain regenerability of Miscanthus ecallus for a long enough period of time to allow transformation and regeneration. Alternatively, in a preferred method, one can employ chlorophyll synthesis as a marker for the visual identification of regenerable ecallus. Using this selection method, the ecallus may be subjected to continuous light for a period of at least about 1 day, 2 days, 3 days, 4 days, 5 days, 6 days or 7 days prior to subculturing ecallus. When subculturing the light-treated ecallus, green ecallus can be preferentially selected, e.g., for regenerability, and transferred. The selected ecallus can optionally undergo further selection steps under like conditions which better insures that the ecallus used in the transformation process is sustainably regenerable embryogenic callus. Even more preferred, the use of chlorophyll biosynthesis as a selection marker can be used in combination with other known means of selection, for example, selection by means of ecallus morphological pattern.

The Agrobacterium-mediated transformation process described herein can be broken into several steps. The basic steps include an infection step (step 1); a co-cultivation step (step 2); a selection step (step 3); and a regeneration step (step 4).

In the infection step, the cells to be transformed are isolated and exposed to Agrobacterium. If the target cells are ecallus, the ecallus is contacted with a suspension of Agrobacterium. As noted above, the Agrobacterium has been modified to contain a gene or nucleic acid of interest. The nucleic acid is inserted into the T-DNA region of the vector. General molecular techniques used herein are provided, for example, by Sambrook et al. (eds.) Molecular Cloning: A Laboratory Manual, 1989, Cold Spring Harbor Laboratory Press, Cold Spring Harbor, N.Y.

Agrobacterium containing the plasmid of interest are preferably maintained on Agrobacterium master plates with stock frozen at about −80° C. As used herein, the term “Agrobacterium capable of transferring at least one gene” refers to Agrobacterium containing the gene or nucleic acid of interest, generally in a plasmid that is suitable for mediating the events required to transfer the gene to the cells to be infected. Master plates can be used to inoculate agar plates to obtain Agrobacterium which is then resuspended in media for use in the infection process. Alternatively, bacteria from the master plate can be used to inoculate broth cultures that are grown to logarithmic phase prior to transformation.

The concentration of Agrobacterium used in the infection step and co-cultivation step can affect the transformation frequency. Likewise, very high concentrations of Agrobacterium may damage the tissue to be transformed and result in a reduced ecallus response. Thus, the concentration of Agrobacterium useful in the methods described herein may vary depending on the Agrobacterium strain utilized, the tissue being transformed, the Miscanthus genotype being transformed, and the like. To optimize the transformation protocol for a particular Miscanthus line or tissue, the tissue to be transformed (ecallus, for example), can be incubated with various concentrations of Agrobacterium. Likewise, the level of marker gene expression and the transformation efficiency can be assessed for various Agrobacterium concentrations. While the concentration of Agrobacterium may vary, generally a concentration range of about 1×10³ cfu/ml to about 1×10¹⁰ preferably within the range of about 1×10⁵ cfu/ml to about 1×10⁹ cfu/ml and still more preferably at about 1×10⁸ cfu/ml to about 1.0×10⁹ cfu/ml will be utilized.

The tissue to be transformed is generally added to the Agrobacterium suspension in a liquid contact phase containing a concentration of Agrobacterium to optimize transformation efficiencies. The contact phase facilitates maximum contact of the cells/tissue to be transformed with the suspension of Agrobacterium. The cells are contacted with the suspension of Agrobacterium for a period of about 10 minutes in MSMO medium. Other equivalent liquid suspensions are known in the art and can be used. See, for example, Ishida et al. (1996) Nature Biotechnol. 14:745-750; EPA 0672752A1; EPA 0687730A1; and U.S. Pat. No. 5,591,616. For example, media containing N6 salts can also be used in the infection step. Murashige and Skoog (MS; (1962) Physiol. Plant 15:473-497) salts include about 1,650.0 mg/l ammonium nitrate, about 6.2 mg/l boric acid, about 332.2 mg/l calcium chloride anhydrous, about 0.025 mg/l cobalt chloride,6H₂O, about 0.025 mg/l cupric sulfate.5H₂O, about 37.26 mg/l Na₂EDTA, about 27.8 mg/l ferrous sulfate.7H₂O, about 180.7 mg/l magnesium sulfate, about 16.9 mg/l manganese sulfate.H₂O, about 0.25 mg/l molybdic acid (sodium salt).2H₂O, about 0.83 mg/l potassium iodide, about 1,900.0 mg/l potassium nitrate, about 170.0 mg/l potassium phosphate monobasic, and about 8.6 mg/l zinc sulfate.7H₂O. Additionally, other media, such as Linsmaier and Skoog (LS; (1965) Physiologia Plantarum 18:100-127) and those set forth in the examples, can be utilized. The macro and micro salts in MS medium are identical to the macro and micro salts in LS medium, but the two media differ in the composition of some of the vitamins and other components (Skirvin (1981) in: Cloning Agricultural Plants Via In Vitro Techniques, Conger, ed., CRC Press, Knoxville, Tenn., pp. 51-140).

In the co-cultivation step, the infected cells prepared as described above are co-cultivated with Agrobacterium. For ecallus, the co-cultivation with the Agrobacterium usually takes place on a solid medium. The ecallus are co-cultivated with the Agrobacterium for about 2 to 5 days, preferably about 4 days. This co-cultivation step preferably takes place in darkness at 20 to 26 degrees C., more preferably about 25 degrees C.

Following the co-cultivation step, the transformed cells may be subjected to a resting step; however, the resting step is optional. Where no resting step is used, an extended co-cultivation step may be utilized to provide a period of culture time prior to the addition of a selective agent.

For the resting step, the transformed cells are transferred to a second medium containing an antibiotic capable of inhibiting the growth of Agrobacterium. This resting phase is performed in the absence of any selective pressures to permit preferential initiation and growth of callus from the transformed cells containing the heterologous nucleic acid. An antibiotic is added to inhibit Agrobacterium growth. Such antibiotics which inhibit the growth of Agrobacterium are known in the art and include cefotaxime, Timentin®, vancomycin, carbenicillin, and the like. Concentrations of the antibiotic will vary according to what is standard for each antibiotic. For example, concentrations of carbenicillin will range from about 50 mg/l to about 500 mg/l carbenicillin in solid media, preferably about 75 mg/l to about 250 mg/l, more preferably about 150 to 200 mg/l. Those of ordinary skill in the art of monocot transformation will recognize that the concentration of antibiotic can be optimized for a particular transformation protocol without undue experimentation. Preferably, no resting step is included.

Following the co-cultivation step, or following the resting step, where it is used, the transformed cells are exposed to selective pressure to select for those cells that have received and are expressing polypeptide from the heterologous nucleic acid introduced by Agrobacterium. Where the cells are ecallus, the ecallus are transferred to plates with solid medium that includes both an antibiotic to inhibit growth of the Agrobacterium and a selection agent. The agent used to select for transformants will select for preferential growth of transformed plant cells within explants containing at least one plant cell into which a selectable marker insert positioned within the binary vector was delivered by the Agrobacterium and stably integrated into the cell's genome.

Generally, any of the media known in the art suitable for the culture of Miscanthus can be used in the selection step, such as media containing N6 salts or MS salts. During selection, the co-cultivated ecalli are cultured for about three weeks, and then surviving or growing ecalli are transferred to fresh selection media for an additional three weeks. After this six week period of time, the selection plates are moved to continuous light, as described above, and ecalli that become green are transferred to “Embryogenic callus regeneration medium” (MECR) supplemented with 150 mg/L Timentin for regeneration of whole plants.

When the transgenic plants are about 1 cm long, they are isolated individually, and moved to fresh MECR supplemented with 150 mg/L Timentin for additional growth and rooting. The rooted transgenic plants are planted in soil and grown to maturity.

Now that it has been demonstrated that Miscanthus can be transformed utilizing Agrobacterium, alterations to the general method described herein can be used to increase efficiency or to transform elite lines which may be inbred lines which may exhibit some recalcitrance to transformation. Factors that affect the efficiency of transformation include the types and stages of tissues infected, the concentration of A. tumefaciens, composition of the media for tissue culture, selectable marker genes, the length of any of the above-described steps involved, kinds of vectors and Agrobacterium strains, and the Miscanthus genotype. Therefore, these and other factors may be varied to determine what is an optimal transformation protocol for any particular Miscanthus genotype. It is recognized that not every genotype will react the same to the transformation conditions and may require a slightly different modification of the protocol. However, by altering each of the variables, an optimum protocol can be derived for any Miscanthus genotype.

While any Miscanthus genotype can be used in the transformation methods described herein, examples of Miscanthus varieties include but are not limited to Miscanthus sinesis.

Further modifications may be utilized including providing a second infection step to increase infection by the Agrobacterium. Also, the vectors and methods described herein can be used in combination with particle bombardment to produce transformed Miscanthus plants. Particle bombardment can be used to increase wounding in the tissues to be transformed by Agrobacterium. (Bidney et al. (1990) Plant Mol. Biol. 18:301-313; EPO486233, herein incorporated by reference). Methods for particle bombardment are well known in the art. See, for example, Sanford et al. U.S. Pat. No. 4,945,050; McCabe et al. (1988) Biotechnol. 6:923-926). Also see, Weissinger et al. (1988) Annual Rev. Genet. 22:421-477; Datta et al. (1990) Biotechnol. 8:736-740; Klein et al. (1988) Proc. Natl. Acad. Sci. USA, 85:4305-4309; Klein et al. (1988) Biotechnol. 6:559-563 (maize); Klein et al. (1988) Plant Physiol., 91:440-444; Fromm et al. (1990) Biotechnol. 8:833-839; Tomes et al. “Direct DNA transfer into intact plant cells via microprojectile bombardment.” In: Gamborg and Phillips (Eds.) Plant Cell, Tissue and Organ Culture: Fundamental Methods; Springer-Verlag, Berlin (1995); Hooydaas-Van Slogteren & Hooykaas (1984) Nature (London), 311:763-764; and Bytebier et al. (1987) Proc. Natl. Acad. Sci. USA 84:5345-5349; all of which are herein incorporated by reference.

After wounding of the cells by microprojectile bombardment, the cells are inoculated with Agrobacterium solution. The additional infection step and particle bombardment may be useful in transforming those genotypes of Miscanthus which are particularly recalcitrant to infection by Agrobacterium.

The following examples are offered by way of illustration and not by way of limitation.

### EXAMPLES

### Example I

### Generation of Transgenic Miscanthus by Seed-Derived Embryogenic Callus

A. Production of regenerable embryogenic callus. Embryogenic callus from “Pure Seed”: Miscanthus sinensis variety “Pure Seed” seed (obtained from Jelitto, Staudensamen, Germany) was sterilized by immersion in 20% bleach (plus 0.1% Triton-X100®) for 20 minutes, followed by five rinses in sterilize distilled water. All manipulations after the sterilization steps were performed in an aseptic manner in a laminar air flow cabinet. The sterilized seeds were plated onto Miscanthus Embryogenic Callus Induction and Growth medium (MECG) in Petri dish plates (100×25 mm) and sealed with three layers of saran wrap. The plates were incubated in continuous darkness at 29° C. for 6 weeks. High quality embryogenic callus was visually selected under the dissection microscope at this time.

B. Culture of regenerable embryogenic callus. Once the embryogenic callus was obtained, the embryogenic callus was incubated on MECG in continuous darkness at 29° C. The ecallus was routinely subcultured to fresh MECG approximately every three to four weeks. The plates containing the ecallus cultures were incubated at 29° C. for 3-7 days in continuous white light provided by cool white florescent tubes (70 μmol m−2 s−1) prior to subculture. The exposure to light induced some parts of the ecallus to turn green. The ecallus selected for subculture was preferentially selected based on color (green ecallus was selected) in combination with morphological pattern. As such, chlorophyll biosynthesis was used as a selection marker for regenerable ecallus maintenance.

C. Infection and Co-cultivation of the embryogenic callus with Agrobacterium tumefaciens. Transformation of the ecallus described above was initiated by infection and co-cultivation with Agrobacterium tumefaciens strain GV3101 (pMP90) harboring the PBI121 binary vector. Agrobacterium strain GV3101 is a strain that contains gentamicin resistance on the Ti vir plasmid and kanamycin resistance on the binary plasmid. The PBI121 binary vector also carries the β-glucuronidase (GUS) as a reporter gene, and neomycin phosphotransferase II (NPTII) gene conferring G418 (Geneticin) resistance as a selectable marker within the T-DNA.

The above agrobacteria was grown in 50 ml LB liquid Agrobacterium growth medium (plus 100 ppm Kanamycin) in a 250 ml Erlenmeyer flask at 250 rpm at 28° C. overnight. Young Miscanthus sinensis “Pure Seed” ecallus grown on MECG at a size of about three millimeters in diameter were selected under a dissection microscope. The stage of growth of the ecallus used for co-cultivation was immediately following exposure to light. The selection was accomplished using ecallus morphological patterns combined with chlorophyll biosynthesis as a selection marker for regenerable ecallus. The agrobacteria grown overnight was diluted to OD 0.6 with MECG liquid medium. The selected ecallus was infected by immersion in the Agrobacterium liquid for 5-10 minutes in a Petri dish. The Agrobacterium liquid was removed by sterile pipette. The ecallus was then transferred on to “Agrobacterium and embryogenic callus co-cultivation medium” (MECC) in Petri dish plates for co-cultivation. The plates for co-cultivation were incubated in the dark at 25° C. for five days.

D. Culture and selection of transformed Miscanthus embryogenic callus. After five days of co-cultivation, ecalli were transferred onto ““Transgenic embryogenic callus selection medium” (MECS; MECG plus filter sterilized 100 ppm G418 (Geneticin) for NPTII gene selection, and 150 ppm Timentin to eliminate agrobacteria). The culture was incubated in continuous darkness at 29° C. After three weeks, the ecallus was transferred to fresh MECS under the same growth conditions. After another three weeks, the newly formed ecallus was assayed for the presence of GUS, and the transformed ecallus turned blue in color when exposed to the GUS stain solution within a few hours.

E. Regeneration of whole transgenic plants from the co-cultivated embryogenic callus. After six weeks selection on MECS in the dark, the plates were moved to continuous white light provided by cool white florescent tubes (70 μmol m−2 s−1) for 2 weeks. The green plants that formed were transferred to MECR supplemented with 150 mg/L Timentin medium for further growth and rooting. The leaf tip of a regenerated whole Miscanthus sinensis plant was assayed for the presence of GUS. The transformed Miscanthus sinensis plant leaf tip turned blue in color when exposed to the GUS stain solution within a few hours. A whole, rooted Miscanthus sinensis plant was planted in a pot filled with autoclaved “sunshine” soil mixture, and the pot was kept in a growth room with temperature and light control for acclimatization and growth.

### Example II

### Generation of Transgenic Miscanthus using Immature Inflorescence (Spikelet)-Derived Embryogenic Callus

Embryogenic callus from immature inflorescence: Miscanthus sinensis seed (variety “Late Hybrid” obtained from Jelitto, Staudensamen, Germany) was planted and grown in soil. When the tillers had grown to the point where 5-6 full open leaves were present, the top internode was harvested and sterilized with 20% Bleach (plus 0.1% Triton X100) for five minutes, followed by five rinses in sterilized distilled water. The immature spikelets were removed from the top internode and plated on MECG media in Petri dish plates (100×25 mm) and sealed with three layers of saran wrap. The plates were incubated in continuous darkness at 29° C. for 6 weeks, at which time embryogenic callus was present on many of the explants. High quality embryogenic callus was visually selected under the dissection microscope at this time.

The selection of regenerable ecallus, co-cultivation with Agrobacterium and subsequent selection of transformed ecallus were performed as described in Example 1 above (parts B through D). Transformed ecalli were obtained.

The regeneration of the ecallus and production of transgenic Miscanthus plants are achieved by the method described in Example 1 above, Part E.

### Example III

### Generation of Transgenic Miscanthus with Sulfonamide Herbicide Resistance

Transgenic Miscanthus plants that are resistant to sulfonamide herbicide are produced using the methods described herein. This is accomplished by selecting a gene that exhibits sulfonamide insensitive activity such as dihydropteroate synthase (DHPS) (see, for example, U.S. Pat. No. 6,121,513, hereby incorporated by reference), selecting an appropriate mitochondrial leader peptide, constructing a fusion construct between the mitochondrial leader and the gene conferring sulfonamide resistance, and inserting the construct in a binary vector to be used in Agrobacterium-mediated transformation of a Miscanthus plant.

The production of ecallus, selection of regenerable ecallus, and co-cultivation with Agrobacterium are performed as described in Examples I and II above. The selection of transformed ecallus is accomplished on a medium such as MECS, but substituting a sulfonamide herbicide such as Asulam for the G418 as the selection agent. Subsequent regeneration of the ecallus and production of transgenic Miscanthus plants are performed as described in Examples I and II above.

Formulations for Media Described Above

The following formulations are for liquid media. If solid media are required, 2.5 g/L Gelrite® may be added before sterilization by, for example, autoclaving.

Basal Medium (MSMO)

MS salts mixture (1×)

Gamborg B5 vitamin mixture (1×)

Maltose (30 g/L)

pH adjusted to 5.7

Agrobacterium Growth Medium (MinA)

Bacto-Tryptone® (10 g/L)

Bacto®-yeast extract (5 g/L)

NaCl (10 g/L)

Embryogenic Callus Induction and Growth Medium (MECG)

MSMO Medium to which is added:

6-Benzylaminopurine (BA) (1.0 mg/L)

2, 4-D (5.0 OR 2.0 mg/L)

Embryogenic Callus Regeneration Medium (MECR)

MSMO Medium to which is added:

0.5 mg/L Gibberellic acid (GA3)

Agrobacterium and Embryogenic Callus Co-Cultivation Medium (MECC)

MSMO Medium to which is added:

6-Benzylaminopurine (BA) (1.0 mg/L)

2, 4-D (5.0 OR 2.0 mg/L)

Acetosyringone (100 μM)

Transgenic Embryogenic Callus Selection Medium (MECS)

MSMO Medium to which is added:

6-Benzylaminopurine (BA) (1.0 mg/L)

2,4-D (5.0 OR 2.0 mg/L)

Timentin (150 mg/L)

G418 (100 mg/L)

All publications and patent applications mentioned in this specification are herein incorporated by reference to the same extent as if each individual publication or patent application was specifically and individually indicated to be incorporated by reference.

The present invention is not limited by the specific embodiments described herein. The invention now being fully described, it will be apparent to one of ordinary skill in the art that many changes and modifications can be made thereto without departing from the spirit or scope of the appended claims. Modifications that become apparent from the foregoing description fall within the scope of the claims.

What is claimed is:

1. A transformed Miscanthus plant derived from embryogenic callus selected for its ability to be grown into a mature plant, wherein the transformed Miscanthus plant comprises a plasmid of interest comprising a recombinant nucleic acid comprising at least one recombinant T-DNA border sequence incorporated into its genome.

2. The transformed Miscanthus plant of claim 1, wherein the cultured embryogenic callus is selected using chlorophyll synthesis as an indicator of the ability of embryogenic callus to develop into a mature plant.

3. The transformed Miscanthus plant of claim 1, wherein the transformed Miscanthus plant comprises fewer than 5 copies of the recombinant nucleic acid incorporated in its genome.

4. The transformed Miscanthus plant of claim 1, wherein the recombinant nucleic acid further comprises a polynucleotide that confers resistance to a selection agent.

5. The transformed Miscanthus plant of claim 4, wherein the polynucleotide encodes the neomycin phosphotransferase II (NPTII) enzyme.

6. The transformed Miscanthus plant of claim 4, wherein the recombinant nucleic acid comprises a second polynucleotide that confers herbicide tolerance.

7. A transformed tissue of the transformed Miscanthus plant of claim 1.

8. A transformed seed produced by the transformed Miscanthus plant of claim 1, wherein the transformed seed comprises the plasmid of interest.

9. A Miscanthus progeny plant derived from the transformed seed of claim 8, wherein the progeny plant comprises the plasmid of interest.

10. A transformed plant cell derived from the plant of claim 1.

11. A method for preparing a transgenic Miscanthus plant transformed with a plasmid of interest comprising a recombinant nucleotide sequence, the method steps comprising:
(a) selecting an embryogenic callus that can be grown into a mature plant, where the embryogenic callus is derived from a target Miscanthus plant, and the selecting includes analysis of chlorophyll synthesis as an indicator of the ability of the embryogenic callus to develop into a mature plant;
(b) contacting the embryogenic callus with an Agrobacterium comprising the plasmid of interest;
(c) co-cultivating the embryogenic callus with the Agrobacterium to produce a transformed embryogenic callus;
(d) growing the transformed embryogenic callus into the transgenic Miscanthus plant.

12. The method of claim 11, wherein the selecting comprises exposure of the embryogenic callus to continuous light for a period of at least one day.

13. The method of claim 11, wherein the selecting comprises exposure of the embryogenic callus to continuous light for a period of at least two days.

14. The method of claim 11, wherein the recombinant nucleotide sequence comprises a polynucleotide that confers resistance to at least one selection agent.

15. The method of claim 11, wherein the transgenic Miscanthus plant comprises the plasmid of interest and at least one other vector.

16. The method of claim 11, wherein the selection step of step (a) includes analysis of chlorophyll synthesis and morphological examination as an indicator of the ability of the embryogenic callus to develop into a mature plant.

17. A transformed Miscanthus plant produced by:
(a) preparing embryogenic callus from a target Miscanthus plant, wherein the embryogenic callus has a genome and is selected for its ability to be grown into a mature plant;
(b) providing an Agrobacterium comprising a plasmid of interest comprising a recombinant nucleotide sequence, wherein the recombinant nucleotide sequence comprises a polynucleotide of interest;
(c) contacting the embryogenic callus with the Agrobacterium;
(d) co-cultivating the embryogenic callus with the Agrobacterium for a sufficient time for the a polynucleotide of interest to integrate into the genome of the embryogenic callus to form transgenic Miscanthus callus; and
(e) growing the transgenic Miscanthus plant from the embryogenic callus of step (d), wherein the transgenic Miscanthus plant comprises at least one copy of the a polynucleotide of interest incorporated in its genome.

18. The transformed Miscanthus plant of claim 17, wherein the embryogenic callus of step (a) is selected using chlorophyll synthesis as an indicator of ability of the embryogenic callus to develop into a mature plant.

19. The transformed Miscanthus plant of claim 17, wherein the recombinant nucleotide sequence further comprises a second polynucleotide that confers resistance to a selection agent, and the second polynucleotide is integrated into the genome of the embryogenic callus during the co-cultivation step of (d).
